(19) United States
(12) Patent Application Publication
(10) Pub. No.: US 2008/0217425 A1
(43) Pub. Date: Sep. 11, 2008

(54) Evaporating Device

(75) Inventors: Martin Butler, Hull (GB); Laurie Labesque, Parsippany, NJ (US)

Correspondence Address: NORRIS, MCLAUGHLIN & MARCUS, 875 THIRD AVE, 18TH FLOOR, NEW YORK, NY, 10022, US

(73) Assignee: RECKITT BENCKISER (UK) LIMITED, Slough, Berkshire (GB)

(21) Appl. No.: 12/065,351

(22) PCT Filed: Aug. 29, 2006
(86) PCT No.: PCT/GB06/03194

§ 371 Date: Feb. 29, 2008

(30) Foreign Application Priority Data
Sep. 8, 2005 (GB) 0518308.2

(51) Int. Cl. A61L 9/12

(52) U.S. Cl. 239/45

(57) ABSTRACT

An evaporating device comprising:
a container body, comprising a base, wall(s) and a neck, which further comprises a solution of an active composition; and
a plurality of porous wicks
   wherein:
at least part of each porous wick is inside the container; and at least part of each porous wick protrudes upwards from the container
   characterised in that:
the base of the container comprises an upwardly projecting punt formed such that the lowermost part of each porous wick is always in contact with the solution of active composition during the lifetime of the device.

The present invention relates to an evaporating device for an active composition such as a fragrance or an air freshener, comprising a container and a plurality of porous wicks, wherein the container comprises a punt in its base thereby ensuring that the lowermost portion of each wick is always in contact with the solution of active composition contained within the device container.

Wick-based emanation systems for the dispersal into the air by evaporation vaporised particles of liquids are well known in the art. Such systems are often used in the home with liquids comprising active ingredients such as air fresheners or insecticides. The partially submerged portion of the wick absorbs the liquid containing the active ingredient, some of which diffuses by capillary or wicking action into the exposed, un-submerged portion of the wick. Such devices then use either ambient conditions or heating (usually electrical) to evaporate the active component from the exposed portion of the wick, examples being Airwick Diffuser Actif (Reckitt Benckiser), Glade® (S. C. Johnson). These are single wick devices. Electrical devices which enhance the evaporation rate by means of a fan located in proximity to the wick are also known, for example Mobil′Air®.

There is an increasing demand for more efficient non-electrical devices of this type, which dispense active materials at a slow, substantially constant rate throughout the lifetime of the device. See for example U.S. Pat. No. 5,242,111 or EP0798252. From an environmental as well as an aesthetic point of view, there is as well demand for products which use so-called “natural” materials as far as possible. Such materials are valued for their overall aesthetic effect and minimal environmental impact, but are thus far lacking in efficiency. Devices which simulate the appearance of dried flower and plant arrangements are known, for instance using rattan or twigs inside simple perfume bottles. Such devices are unstable and inefficient as arrangement of wicks in the containers is random and precarious, and the wick will not necessarily be in contact with the solution to be diffused throughout the lifetime of the device.

The objective technical problem to be solved by the present invention therefore is the provision of a more stable and more efficient device for active emanation.

The present invention solves this problem by provision of an evaporating device comprising:
a container body, comprising a base, wall(s) and a neck, which further comprises a solution of an active composition; and
a plurality of porous wicks
   wherein:
at least part of each porous wick is inside the container; and at least part of each porous wick protrudes upwards from the container
   characterised in that:

the base of the container comprises an upwardly projecting punt formed such that the lowermost part of each porous wick is always in contact with the solution of active composition during the lifetime of the device.

In a preferred aspect of the first embodiment of the invention as hereinbefore described, preferably the punt will be concentric with the base of the container with imaginary axis (I) extending through the base and perpendicular to it.

In a further preferred aspect of the first embodiment of the invention as hereinbefore described the punt has an external depth of at least 5% of the overall height of the container body, more preferably punt has an external depth of at least 10% of the container body.

In a further preferred aspect of the first embodiment of the invention as hereinbefore described the punt is either hemispherical or punt is frustoconical.

In a particularly preferred aspect of the first embodiment of the invention as hereinbefore described the punt is inwardly extending substantially concave, dome shaped.

The punt optionally has a substantially flattened upper surface.

In a further preferred aspect of the first embodiment of the invention as hereinbefore described the punt is either elliptical or substantially circular in outline.

The punt defines a channel or moat at base of container in which the plurality of wicks are positioned. Preferably the depth of the moat is between 5% and 10% of the overall height of the container body, and the width of moat is between 80% and 120% of the diameter of the wicks. In a particularly preferred embodiment of the present invention, the width of the moat is substantially equal to the diameter of wicks.

The slope of the walls of punt will be chosen to be sufficiently steep so as to lead the wicks to sit in the moat.

The punt also strengthens the bottom of the container making it less fragile and more stable to tipping by lowering the centre of gravity of the container.

In use, the wicks will be substantially symmetrically arranged, thus ensuring a well balanced and stable device. In particular, even when a minimal amount of active containing composition remains, the bottom of the wicks will still be in contact with the solution, thereby ensuring maximum lifetime and minimum environmental impact from disposal.

The porosity and rigidity of the wicks are also important factors to take into consideration when choosing the number and dimensions of the wicks, and therefore wicks will comprise synthetic materials or mixture of natural and synthetic as appropriate or desired. The exposed surface area of each wick determines the rate and efficiency of diffusion of the active ingredient, and this is determined by (i) wick porosity and (ii) the amount of wick which protrudes into atmosphere from container.

The present invention as hereinbefore described allows a higher ratio of length of wick (outside: inside) than usual, thereby also maximising the efficiency of the device.

In a further preferred aspect of the first embodiment of the invention as hereinbefore described the wicks comprise natural porous material selected from rattan, bamboo, or synthetic equivalents thereof.

In a particularly preferred aspect of the first embodiment of the invention as hereinbefore described there are between 4 and 25 porous wicks, more preferably between 8 and 20 porous wicks, especially preferably between 12 and 16 wicks.

Preferably the length of each wick is at least 125% of the overall height of the container body, more preferably at least 150% of the overall height of the container body, especially preferably at least 175% of the overall height of the container body.

Preferably the ratio of the portion of each wick outside the container to the portion of each wick inside the container is at least 0.5:1, more preferably at least 0.75:1, especially preferably at least 1:1.

In a yet further preferred aspect of the first embodiment of the invention as hereinbefore described the active solution comprises an active component selected from a fragrance, an odour masking agent, a sanitizer, a medicament or an insecticide and optionally a solvent selected from water, an alcohol, a hydrocarbon or a mixture thereof. Preferably the active composition comprises a fragrance.

Preferably the active component is present in the active composition in a concentration range of at least 1%, preferably at least 20%, more preferably at least 50%, especially preferably at least 80% (all % figures are w/w).

In a particularly preferred aspect of the first embodiment of the invention as hereinbefore described the active composition contains <5 wt % of water.

In another particularly preferred aspect of the first embodiment of the invention as hereinbefore described the active composition comprises <5 wt % of a non-aqueous solvent.

In a yet further preferred aspect of the first embodiment of the invention as hereinbefore described the active composition is coloured and comprises a dye soluble in the composition.

In another preferred aspect of the first embodiment of the invention as hereinbefore described the container the container is clear, transparent or opaque.

Preferably the container comprises glass or plastic.

Preferably the shape of the container is a standard bottle, which may be cylindrical, ovoid, square or other polygonal in profile. Preferably the size of the container corresponds to standard sized perfume containing bottles, with overall height from 5 cm to 50 cm, base dimensions 2 to 10 cm, and internal volumes from 5 to 100 ml.

Preferably the solution of active composition in the container is sealed by a closed cap before product use. In a yet further particularly preferred aspect of the first embodiment of the invention as hereinbefore described the neck of the container comprises a screw thread. In this embodiment the container, in use, further comprises a collar engaged with the screw thread, which is not only decorative but also optionally serves to protect the device before start of use by addition of a peelable impermeable seal to the top of the collar across the opening of the container. Removal of the peelable seal followed by insertion of the wicks through the opening makes the device ready for use.

In an especially preferred embodiment of the present invention, in order to avoid spillage of the active containing composition on accidental inversion of the container, there is provided an open cap. Anti-spill devices are known in the prior art for containers, see for instance U.S. Pat. No. 6,142,101 and for air fresheners, see for example WO 03/092750, which requires the wick to be sealed into a downwardly projecting sleeve by lagging material, resulting in a complicated single wick arrangement, or U.S. Pat. No. 4,537,351, which requires a complicated multi-part container assembly.

The problem of prevention of spillage in a multiple wick device has been solved in a more straightforward manner in this aspect of the present invention by the use of simple removable open cap.

This particular embodiment of the present invention therefore comprises an evaporating device substantially as hereinbefore described which further comprises an open cap positioned in the opening of the container and engaged with the neck of the container which prevents spillage of the active containing composition.

The cap comprises a first part which is engaged, optionally removeably engaged, with the neck of the container, and a second part projecting downwards through the opening into the container and which serves to reduce the aperture of the opening.

The first and second parts define an internal annular channel at the neck of the container which serves to trap and retain liquid if the container is fully or partially inverted, thereby controlling and substantially preventing spillage.

In a particularly preferred embodiment of this aspect of the invention, the second part of the open cap is frustoconical. In an especially preferred embodiment of this aspect of the invention, the second part of the open cap tapers in a frustoconical manner to reduce the effective diameter of the opening of the container by at least 30%, preferably 40%.

The cap may be made of any material suitable, such as but not limited to plastic, rubber and other resilient non-deformable materials. Preferred is a cap made of plastic.

The engagement of the cap with the walls of the container can be by any means suitable, preferably by annular ridges in the first part of the cap.

In a further particular embodiment of the present invention, the open cap and the collar may form a single component.

In a second aspect of the present invention, there is described a method of evaporating an active substance by use of a device substantially as hereinbefore described, preferably a method which is a method for fragrancing air.

Examples of devices in accordance with the present invention will now be described with reference to the drawings, in which:

FIG. 1 is a perspective view of the container alone;

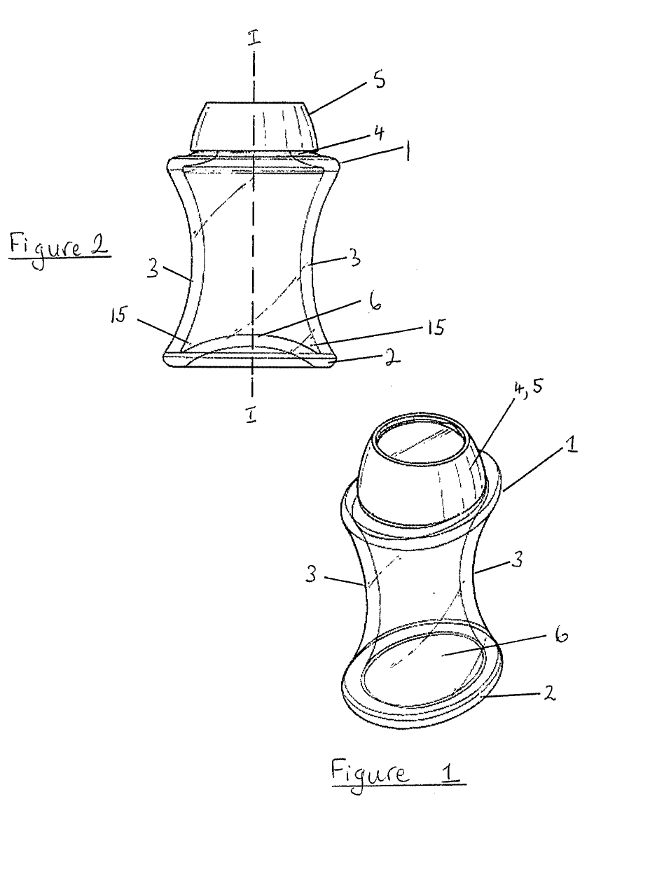

FIG. 2 is a front view of the container alone;

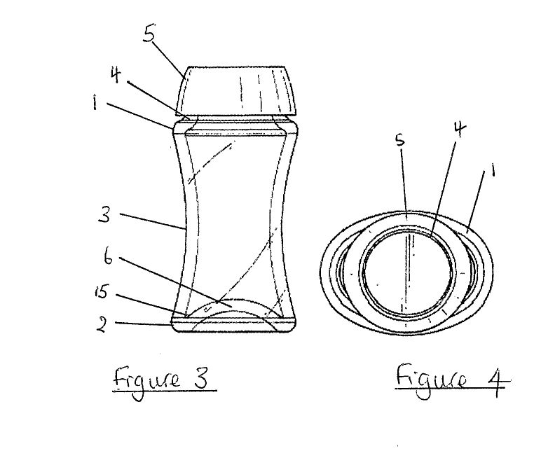

FIG. 3 is a side view of the container alone;

FIG. 4 is a top view of the container alone;

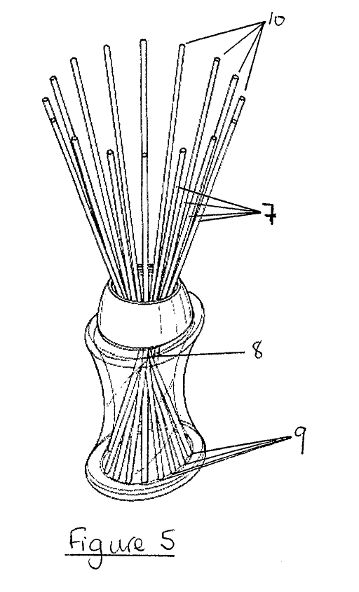

FIG. 5 is a perspective view of the container with wicks positioned as they would be in use;

FIG. 6 is a front view of the container with wicks positioned as they would be in use;

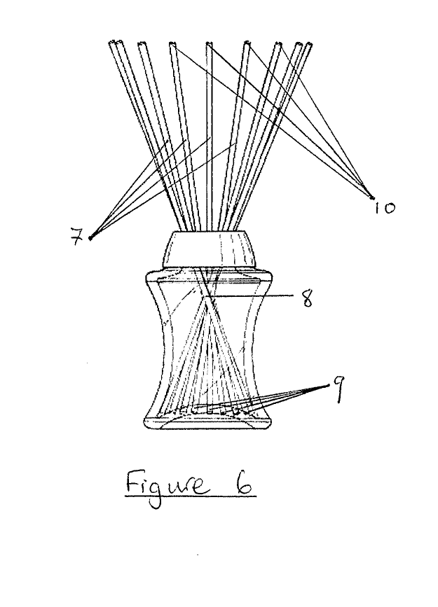

FIG. 7 is a front view of the container, wicks and active composition at beginning of the device lifetime;

FIG. 8 is a front view of the container, wicks and active composition at end of the device lifetime;

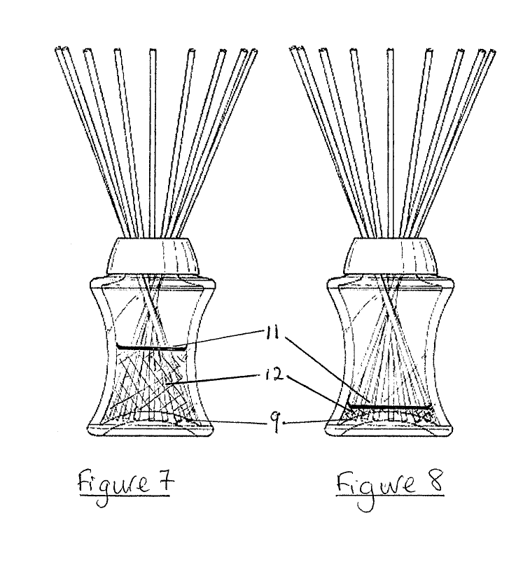

FIG. 9 is a perspective view of the open cap alone;

FIG. 10 is a front view of the cap alone;

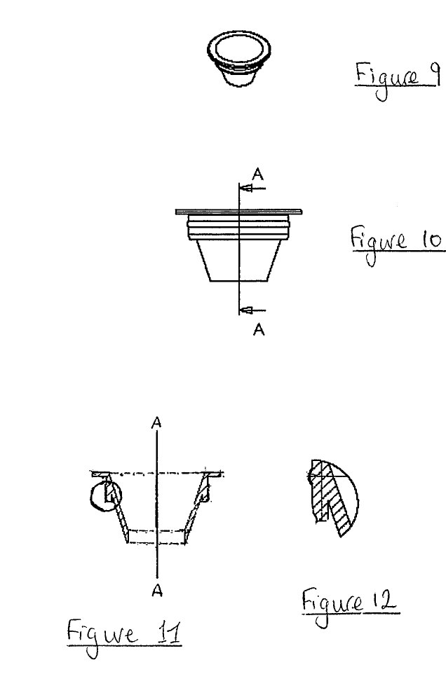

FIG. 11 is a cross section of the front view of cap through axis A; and

FIG. 12 is a cross section of the cap through axis A, showing a close up of the annular channel formed by the first and second parts of the open cap.

FIGS. 1-4 show the container (1), base (2), wall(s) (3), neck (4) with collar (5) engaged by screw thread. Base further has upward pointing punt (6), which defines a channel (15) which runs round the internal perimeter of the base. Container may have any shape but preferred is usually approximately 1.5 times as tall as it is wide, preferably twice as tall as it is wide. In FIG. (2), imaginary axis I is shown, about which the perimeter of the base and the punt are concentric.

FIGS. 5-6 show container with the wicks inserted—here 16 wicks although any number between 4 and 25 could be used. By virtue of choice of size and profile of the recess/punt, the wicks can only distribute themselves in one position, wherein the bottom of each wick (9) sits in channel (15), resulting in symmetrical and stable arrangement as the crossing point of the wicks (“centre of gravity”) just below neck (4).

FIGS. 7-8 show container, wicks and solution of active composition (12), surface level of which is shown (11). In FIG. 7 at start of product lifetime, level situated approx halfway up container, all wicks are submerged in the active containing solution. In FIG. 8, at the end of the product lifetime, when very little active containing solution remains, it can be seen that the ends (9) of the wicks are still in contact with this remaining amount, thus prolonging the active lifetime of the device and minimising the inconvenience to the user of having to dispose of a not completely exhausted container.

FIGS. 9-12 show the anti-spill feature, an open cap. This comprises two conjoined parts, a first part (13) which engages with the inside of the neck/opening of the container and a second part (14) which serves to decrease the diameter of the opening of the container. In the specific embodiment the first part (13) is cylindrical and matches the diameter of the opening of the container, and the second part (14) is frustoconical, tapering from the top of the opening of the container where it has an aperture of equal diameter to that opening, to an aperture of diameter less than that of the opening.

FIGS. 11 and 12 show that where the first and second parts of the open cap meet, at the uppermost part of the open cap, there is formed a channel between the lower portion of the open cap and the side wall of the container, which serves to entrap any liquid composition if the container is upset from an upright position.

1. An evaporating device comprising:
a container body, comprising a base, wall(s) and a neck, which further comprises a solution of an active composition; and
a plurality of porous wicks
wherein:
at least part of each porous wick is inside the container; and at least part of each porous wick protrudes upwards from the container
characterised in that:
the base of the container comprises an upwardly projecting punt formed such that the lowermost part of each porous wick is always in contact with the solution of active composition during the lifetime of the device.

2. An evaporating device according to claim 1 wherein the punt has a depth of at least 5% of the overall height of the container body.

3. An evaporating device according to claim 2 wherein the punt has a depth of at least 10% of the container body.

4. An evaporating device according to claim 1 wherein the punt is either hemispherical or frustoconical.

5. An evaporating device according to claim 1 wherein the punt is inwardly extending substantially concave, dome shaped.

6. An evaporating device according to claim 1 wherein the punt has a substantially flattened upper surface.

7. An evaporating device according to claim 1 wherein the punt is either elliptical or substantially circular in outline.

8. An evaporating device according to claim 1 wherein the punt defines a channel or moat at base of container in which the plurality of wicks are positioned.

9. An evaporating device according to claim 8 wherein the depth of the moat is between 5% and 10% of the overall height of the container body.

10. An evaporating device according to claim 8 wherein the width of moat is between 80% and 120% of the diameter of the wicks.

11. An evaporating device according claim 1 wherein the plurality of porous wicks comprise rattan, bamboo, or synthetic equivalents thereof.

12. An evaporating device according to claim 1 which comprises between 4 and 25 porous wicks.

13. An evaporating device according to claim 12 which comprises between 8 and 20 porous wicks.

14. An evaporating device according to claim 12 which comprises between 12 and 16 porous wicks.

15. An evaporating device according to claim 1 wherein the wick length is at least 125% of the overall height of the container body.

16. An evaporating device according to claim 15 wherein the wick length is at least 150% of the overall height of the container body.

17. An evaporating device according to claim 16 wherein the wick length is at least 175% of the overall height of the container body.

18. An evaporating device according to claim 1 wherein the ratio of the portion of each wick outside the container to the portion of each wick inside the container is at least 0.5:1.

19. An evaporating device according to claim 18 wherein the ratio of the portion of each wick outside the container to the portion of each wick inside the container is at least 0.75:1

20. An evaporating device according to claim 19 wherein the ratio of the portion of each wick outside the container to the portion of each wick inside the container is at least 1:1.

21. An evaporating device according to claim 1 wherein the active composition comprises an active component selected from a fragrance, a sanitiser or an insecticide and a solvent selected from water, an alcohol or a mixture thereof.

22. An evaporating device according to claim 21 wherein the active composition comprises a fragrance.

23. An evaporating device according to claim 21 wherein the active component is present in the active composition in a concentration of at least 1% wt.

24. An evaporating device according to claim 23 wherein the active component is present in the active composition in a concentration of at least 20% wt.

25. An evaporating device according to claim 24 wherein the active component is present in the active composition in a concentration of at least 50% wt.

26. An evaporating device according to claim 25 wherein the active component is present in the active composition in a concentration of at least 80% wt.

27. An evaporating device according to claim 21 wherein the active composition contains <5 wt % of water.

28. An evaporating device according to claim 21 wherein the active composition comprises <5 wt % of a non-aqueous solvent.

29. An evaporating device according to claim 12 wherein the active composition is coloured.

30. An evaporating device according to claim 1 wherein the container is clear, transparent or opaque.

31. An evaporating device according to claim 1 wherein the container comprises glass or plastic

32. An evaporating device according to claim 1 which further comprises an open cap removeably engaged with the neck of the container which prevents spillage of the active containing composition.

33. An evaporating device according to claim 32 wherein the cap comprises an first portion removeably engaged with the neck of the container, and a second portion projecting downwards through the opening into the container and which serves to reduce the aperture of the opening.

34. An evaporating device according to claim 33 wherein the first and second portions define an internal annular channel at the neck of the container.

35. An evaporating device according to claim 34 wherein the second portion is frustoconical.

36. An evaporating device according to claim 35 wherein the second part of the open cap tapers in a frustoconical manner to reduce the effective diameter of the opening of the container by at least 30%.

37. An evaporating device according to claim 36 wherein the second part of the open cap tapers in a frustoconical manner to reduce the effective diameter of the opening of the container by at least 40%.

38. An evaporating device according to claim 32 wherein the open cap is made of plastic, rubber or another resilient non-deformable material.

39. An evaporating device according to claim 38 wherein the open cap is made of plastic.

40. An evaporating device according to claim 32 wherein the engagement of the cap with the walls of the container by is achieved by annular ridges in the first part of the cap.

41. An evaporating device according to claim 32 wherein the open cap and the collar form a single component part.

42. (canceled)

43. A method of evaporating an active substance by use of a device according to claim 1.

44. A method according to claim 43 which is a method for fragrancing air.

45. (canceled)
